(19) United States
(12) Patent Application Publication
(10) Pub. No.: US 2010/0262060 A1
(43) Pub. Date: Oct. 14, 2010

(54) Rigid, flexible, non-touching adhesive or tape fastened bandages, covers and protectors for scars, lacerations, wounds, infections, etc.

(76) Inventor: Tanner Marvin George, SR., Memphis, TN (US)

Correspondence Address: Tanner M. George , Sr., 1268 Lapaloma Street, Memphis, TN, 38114, US

(21) Appl. No.: 12/284,525

(22) Filed: Sep. 23, 2008

(51) Int. Cl. A61F 13/02

(52) U.S. Cl. 602/54

(57) ABSTRACT

Present scar, lacerations and other wound bandages and dressings are cloth types that directly engage an affected area. Tape to hold present bandages or dressings in place is attached to a bandage or dressing and the surrounding skin. If medication is applied to a wound, direct contact bandages or dressings contact that medication. The invention covers and adheres to skin that surrounds a wound without directly contacting that wound. The rigidity and flexibility of the invention allows it to adjust to body contours. The invention does not disturb either the wound or the medication that is applied to the wound because it does not touch the wound or scar. Additionally, the invention protects the wound from the surrounding environment such as clothing or inadvertent bumps.

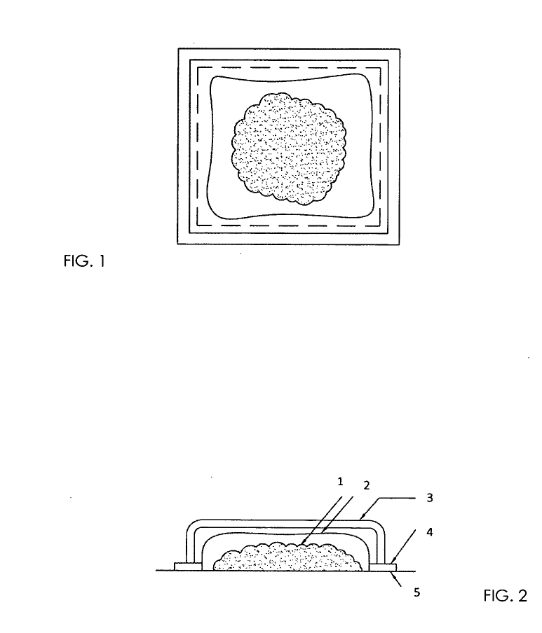

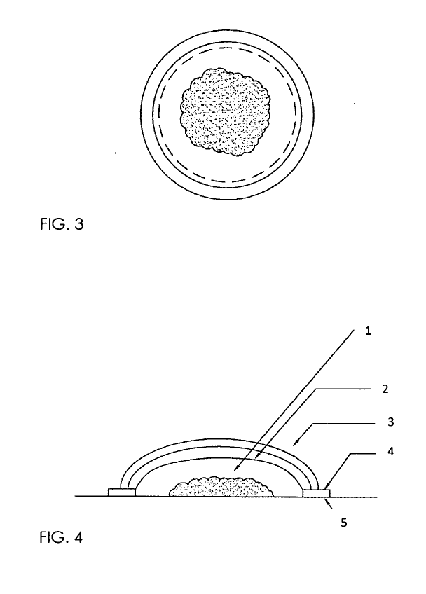

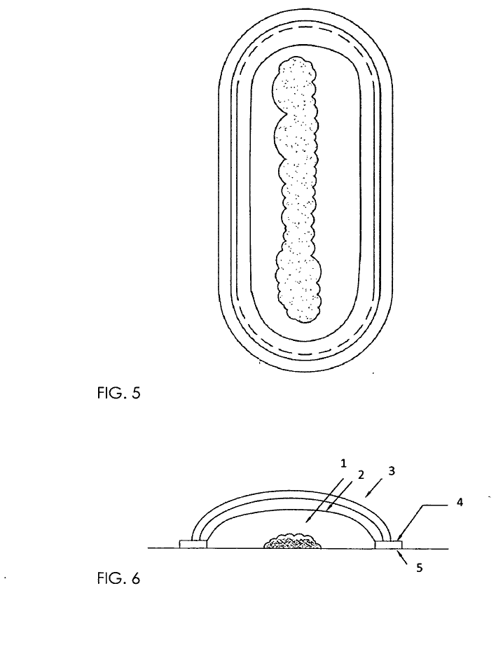

### CROSS-REFERENCE TO RELATED APPLICATIONS

USPTO, Google and Wickipedia searches did not reveal any related applications.

### FEDERALLY SPONSORED RESEARCH OR DEVELOPMENT

No Federally sponsored research or development associated with this invention.

### REFERENCE TO SEQUENCE LISTING

No table, or computer program listing Compact Disc Appendix is included.

### BACKGROUND OF THE INVENTION

The field of endeavor to which the invention pertains is medical in terms of providing rigid, flexible non-touching adhesive or tape fastened covers for scars, lacerations, wounds, infections, etc., that are sensitive to the touch of traditional bandages and bandage covers.

The invention falls in the USPTO category titled: BANDAGE STRUCTURE, #602,42,128, sub-titled: surgery, splint, brace or bandage for skin laceration or wound cover.

The invention resulted from conversations with patients in heart surgeons' and heart specialists' offices who complained that traditional bandages and even pajamas, gowns, and other clothing caused pain when in contact with surgical scars. No references to rigid, flexible non-touching adhesive or tape fastened covers for scars, lacerations, wounds, infections, etc. were found in traditional bandage makers' literature whose products require cloth to touch the skin.

### BRIEF SUMMARY OF THE INVENTION

The invention provides a protective cover over a sensitive skin scar, laceration, infection or other wound. A wound may be dressed with medication or have a bandage covering the wound. The invention will simply adhere to the skin around the wound. It will not interfere with the wound. The invention will protect the wound from extraneous cloth rubbing against either the wound or the bandage.

### BRIEF DESCRIPTION OF THE SEVERAL VIEWS OF THE DRAWING

Three drawings; 1A, 2A and 3A describe the invention. Each drawing contains a Top or Plan View and a Front View.

Drawing 1A depicts a boxed bandage and cover.

Drawing 2A shows a rounded bandage and cover.

Drawing 3A depicts a semi-circle bandage and cover. Each of the drawings show the invention covering a scar or wound.

### DETAILED DESCRIPTION OF INVENTION:

The invention is designed to be placed over a skin scar, laceration, abrasion, infection or other wound for the purpose of providing a rigid flexible bandage protective cover for the wound. The invention does not touch the wound. Pursuant to drawings 1A, 2A and 3A, the material used by the invention is light weight rigid flexible Styrofoam, wood, or plastic of the type that is used for drinking and eating purposes.

These types of material have been proved to be safe for human contact. They are used by the invention for safety because the invention engages the skin.

The invention is distinguished from the cloth and paper type bandages and dressings that touch the skin. This inventor recently underwent open heart surgery. His chest scar and leg scar are sensitive to touch. Cloth and paper bandages severely irritated the wounds and caused pain. Clothing that touched the wounds also irritated the wounds and caused pain. Placing a non-touch bandage, as detailed in drawing 1A and 3A over the scars alleviated the irritation. Better control and adherence of medication to the wounds were also noted with the non-touch bandage.

The invention's housings are fabricated by extrusion or assembly to conform to the shapes as detailed in drawings 1, 2 and 3. Each of the three shapes may vary in size from less than one square inch to more than one square foot. Height may also vary depending on the shape of the skin over which the bandage cover is installed. This is based on the type, location and size of the wound to be covered.

Adhesive or paper tape is installed completely around the bottom of the bandage to form a bandage foot. The adhesive is of the type presently widely used in over the counter bandages. The adhesive may be installed by present machines that apply adhesive to paper and cloth bandages. Paper of the type presently used in over the counter bandages to protect adhesive will be placed over the invention's adhesive or paper tape feet for adhesive protection. Peeling this paper from the foot of the invention will be required in order to apply the bandage cover.

Where a user wishes to remove parts of the foot to gain more bandage or cover flexibility, the user may cut small plugs from the bandage foot. The design is such that the bandage cover may be placed over any wound with one hand after removing the protective adhesive paper by simply plopping it over a wound.

1. I, Tanner M. George, Sr. hereby claim that I am the sole inventor of the items titled: Rigid, Flexible, Non-Touching Adhesive Or Tape Fastened, Boxed, Round, and Semi-Circle Bandage and Cover as depicted on drawings 1, 2, and 3 of the Brief Description of the Several Views of the Drawing of this Specification.
